(19) United States
(12) Patent Application Publication
(10) Pub. No.: US 2006/0224027 A1
(43) Pub. Date: Oct. 5, 2006

(54) Shaped catalyst body, particularly for use as catalysts in hydrogenation

(76) Inventors: Thomas Turek, Goslar (DE); Aurel Wolf, Wulfrath (DE); Christian Munnich, Leverhusen (DE); Rainer-Leo Meisel, Laufenburg (DE); Stefan Zimmermann, Laufenburg (DE)

Correspondence Address: BAYER MATERIAL SCIENCE LLC, 100 BAYER ROAD, PITTSBURGH, PA, 15205, US

(21) Appl. No.: 11/368,800

(22) Filed: Mar. 6, 2006

(30) Foreign Application Priority Data
Mar. 8, 2005 (DE) 102005011047.9

(51) Int. Cl. C07C 5/03; B01J 21/04; B01J 23/02

(52) U.S. Cl. 585/276; 502/439

(57) ABSTRACT

The present invention relates to a shaped catalyst body, particularly for use as catalysts in hydrogenation.

The present invention relates to a shaped catalyst body, particularly for use as catalysts in hydrogenation.

Heterogeneous hydrogenation catalyst based on nickel and other elements or optionally further elements suitable for hydrogenation, such as Co or Cu, and at least one further catalytically inactive metal, in particular aluminium, are employed for the hydrogenation of organic compounds. These so-called Raney catalysts generally require an activation step, in which the catalytically inactive metal is removed by leaching. Raney catalysts are generally used as a fine powder, which, although it leads to a high activity, nevertheless makes separation from the reaction mixture time-consuming and therefore expensive. For instance, sugars such as glucose are technically hydrogenated heterogeneously with powdered catalysts to give consecutive products, for example sorbitol. The hydrogenation is carried out batchwise in stirred reactors and the powdered catalyst then needs to be elaborately separated.

Shaped catalyst bodies (tablets, pellets, extrudates etc.) are used as an alternative to powdered Raney catalysts, for example in continuously operated trickle bed reactors. However, these shaped bodies with an average size of approximately 1 to 10 mm have the disadvantage of low activity and nonuniform wetting during the reaction. The rate constant of the catalytic reaction, normalised to the mass of catalyst, is thus relatively low since the reaction takes place almost exclusively on the tablet surface, whereas the majority of the tablet mass inside the tablet is not involved in the reaction (diffusion limitation).

The economic utilisation of expensive catalyst metals in tablet catalysts is relatively limited for this reason.

EP-A-0094577 discloses the production of an electrode using plasma spraying methods, by applying a nickel underlayer (30 to 60 μm) on a support of soft iron or steel and subsequently applying a 20 to 60 μm thick Raney layer of nickel and aluminium. This method entails the disadvantage of having two steps. Another disadvantage is that a mixture of Ni and Al powders is used instead of a Ni—Al alloy powder, so that formation of the required Ni—Al phases is not guaranteed. EP-A-0100659 also describes the production of a cathode with low hydrogen overvoltage for chloralkali electrolysis. The cathode is produced by applying a Ni/Al alloy with a specific particle fraction by means of plasma spraying onto an electrically conductive and porous metal (Fe and alloys). The layer thicknesses achievable with this method are in the range of from 13 to 508 μm. With >56 wt. %, the composition used for the Ni/Al alloy has significantly more than the Ni contents normally used for catalysts. This is because the solubility of aluminium decreases with an increasing Ni concentration, so that it is difficult to leach the aluminium out during activation. In principle, therefore, the person skilled in the art cannot expect that such materials conceived for chloralkali electrolysis will also be suitable as catalysts. Publications concerning electrode production do not therefore contain any indications about catalyst production, and vice versa.

EP-A-0120122 discloses a method for hydrogenating plant oils. The method uses a catalyst with a mesh structure, in which there is a Raney nickel layer on a nickel alloy layer. The catalyst is produced by coating aluminium onto the surface of a nickel alloy mesh which contains a promoter, heating the coated mesh surface to from 660° to 880° C. so that part of the aluminium enters the outer region of the nickel alloy mesh, a crystalline alloy layer being formed which primarily comprises the beta structure in its outer region, and leaching the aluminium out to form a Raney metal layer. This catalyst has various disadvantages. If the support itself consists of nickel or a nickel alloy, then large parts of the nickel remain unused. If the support does not consist of nickel, then nickel must first be elaborately applied as an outer layer on the support. In both cases, only then is it possible to apply the aluminium which is converted into the desired alloy layer in a further step. Even then, however, sizeable parts of the nickel inside the outer layer remain unused. EP-A-0091027 describes the use of this catalyst for hydrogenating aromatics, EP-A-0091028 describes the use of this catalyst for hydrogenating aromatic amines and EP-A-0087771 describes the use of this catalyst for hydrogenating carbon monoxide or carbon dioxide.

U.S. Pat. No. 3,637,437 discloses self-supporting Raney silver or Raney of the nickel layer structures for use as electrode material. They may be coated onto nickel foil. The geometry of the structures is not explained in detail. An alloy of a Raney metal is applied onto a metal substrate by plasma spraying, the spraying parameters being adjusted so that the particles are not fully melted, in order to produce a porous layer. In order to achieve the required mechanical stability, however, measuring 0.1 to 2 mm the layers produced are very thick. A large quantity of the expensive Raney metal is therefore needed for production, which makes the method economically not very attractive. Furthermore, owing to their thickness and the special structure typical of thermally sprayed layers, the materials are not very flexible. Further processing to form curved or rolled packing is therefore not possible without damaging the layer.

Another method for producing supported Raney catalysts is described in JP 63044944. This method involves the application of Al and Ni by plasma spraying methods in layer thicknesses of 30 to 40 μm onto an interlayer of Al2O3. The bonding between metal and oxide ceramic materials, however, is generally much less than with metal-metal contacts. Furthermore, the multistage method is very elaborate. A significantly lower flexibility is achieved owing to the ceramic interlayer.

WO 01/47633 describes the production of Raney catalysts by alternately applying thin layers of nickel and aluminium by means of electron beam evaporation. The production is preceded by a heat treatment of the support at 700 to 1100° C. in an atmosphere containing oxygen. The layer thicknesses are 0.01 to 100 μm. Disadvantages here, in particular, are the elaborate fabrication of the individual layers and the energy-intensive pre-treatment of the support. Foils, knits or fabrics are used as supports.

Application of the Raney material by means of evaporating or atomising the liquid metal component with a gas is described in WO 01/76737. A reaction of the support with the applied metals is intended to take place. The optimal properties of the resulting interlayer depend crucially on the temperature of the support. Elaborate temperature regulation is therefore necessary. Layer thicknesses of 250 to 550 μm can be obtained by these methods.

JP 2002204957 describes the production of a Raney catalyst using the following steps: producing Ni/Al powder with a defined particle size (44 μm), dispersing the powder to form an aqueous Ni/Al/polyvinyl alcohol suspension, coating a metal support (wire mesh) and subsequently sintering at 1200° C. Layer thicknesses of 5 to 200 μm are obtained by this method. The layer production is carried out using many elaborate manufacturing steps, resulting in high production costs.

It is therefore an object of the inventors to provide an easily produced alternative to tabletted Raney catalysts, with which continuous hydrogenation methods can be made cost-effective. In a fixed bed arrangement, the catalysts should at the same time achieve rate constants comparable with powder catalysts, when normalised to the mass of catalyst. In particular, the catalyst should furthermore be suitable for the hydrogenation of carbohydrates.

The object is achieved according to the invention in that the active Raney metal is present has a thin layer on a suitable substrate. The layers are produced by the thermal spraying and cold-gas spraying methods. Catalyst packing, which can be used in continuously operated hydrogenation reactors, is preferably constructed from the coated substrates. In a fixed bed arrangement, owing to the thin catalyst layer and the large geometrical surface area, these catalysts achieve rate constants comparable with powder catalysts, when normalised to the mass of catalyst.

The invention therefore provides shaped catalyst bodies, obtainable by a method which comprises thermally spraying at least one catalytically active metal and at least one catalytically inactive metal onto a support and subsequently removing the inactive metal(s).

The thermal spraying method as used according to the invention is in particular the spraying method specified in DIN 32350.

In a preferred embodiment, the thermal spraying is selected from the group of methods which consists of: flame spraying, for instance high-speed flame spraying; detonation spraying; plasma spraying, for instance atmospheric plasma spraying or low-pressure plasma spraying; laser spraying; arc discharge spraying and cold-gas spraying. The thermal spraying is particularly preferably carried out by high-speed flame spraying, atmospheric plasma spraying or cold-gas spraying.

In thermal spraying, a generally powdered or wire-shaped spraying material is fully or partially melted in a gun by supplying energy. The sprayed particles are then projected by a high-speed gas jet onto the component to be coated. When they impact on the surface to be coated, the particles flatten out while adapting their shape to the surface and rapidly cool. The subsequently arriving particles thus form a lamellar layer structure. The bonding of the particles on the substrate and with one another is in this case based on mechanical fixing, adhesion and chemical-metallurgical interactions. The various thermal spraying methods differ in respect of the way in which the spraying material is heated and accelerated. Different speeds and temperatures of the sprayed particles consequently result according to the spraying method.

In a particularly preferred embodiment, the catalytically active layer is produced by plasma spraying. It is in plasma spraying that the highest process temperatures, up to 25,000° C., are produced by generating an ionised gas (plasma).

On the other hand, the highest particle speeds and therefore the densest and best-bonding layers can be achieved with high-speed flame spraying, which is why this method is likewise preferred according to the invention. In this method, a fuel-oxygen mixture is burnt in a combustion chamber at a high pressure but much lower temperatures than in plasma spraying. The combustion gases, and the powder particles incorporated into them, are then accelerated to very high speeds in a nozzle.

In contrast to the methods described above, the process temperatures in cold-gas spraying are so low that no melting of the coating powder takes place. By the expansion of a gas (generally nitrogen or helium) which is at a very high pressure, the particles are accelerated to speeds so high that they become fixed and partially welded together by their high kinetic energy without melting. A prerequisite for this is that the coating material must have suitable mechanical properties. This is true of most metal alloys.

According to the invention, the best results are achieved by spraying an alloy of catalytically active and/or inactive metal by means of plasma spraying, high-speed flame spraying or cold-gas spraying. Layers are obtained which are flexible but nevertheless stable, which withstand the formation of curved structures without damage and can be activated to form highly active hydrogenation catalysts.

According to the invention, the catalytically active metal is preferably at least one catalytically active metal which can be used in a heterogeneously catalysed reaction. It is preferably selected from the group which consists of: nickel, silver, copper, cobalt, ion, ruthenium, palladium and platinum. These metals are capable of forming so-called Raney catalysts in which a catalytically inactive metal alloyed with them, as described below, is leached from the alloy.

In a particularly preferred embodiment, nickel or cobalt constitutes the catalytically active metal. Nickel is particularly preferred.

In another possible embodiment, at least one promoter metal, which at least partially remains as an active quantity in the catalytically sprayed active layer after the catalytically inactive metal is removed, may be sprayed in addition to the catalytically active metal.

Examples of such promoters include conventional promoters for Raney catalysts.

In a particularly preferred embodiment, the catalytically active metal and the catalytically inactive metal, and optionally a promoter metal other than these metals, are sprayed in the form of an alloy. This has the advantage that, in contrast to the application of metal powders separated from one another, intermetallic phases are formed so that very finely divided or porous catalytically active layers with a large surface area are obtained after leaching.

The atomic ratio of the catalytically active metal and the catalytically inactive metal depends on the type of catalytically active and catalytically inactive metal and the intermetallic phases formed from them. In general, the atomic ratio of catalytically active to inactive metal is preferably approximately 50:50 to 10:90, preferably 40:60 to 20:80. The atomic ratio of catalytically active to inactive metal is preferably from 35:65 to 25:75. In the case of using nickel and aluminium, as one of the preferred embodiments, the atomic ratio of nickel to aluminium is preferably approximately 50:50 to 10:90: The weight of the catalytically active metal, in particular nickel, in relation to the total amount of as yet unactivated catalyst layer applied, is preferably between 20 and 60 wt. %, preferably between 45 and 55 wt. %.

The layer thickness of the sprayed catalytically active layer is preferably less than 100 μm, more preferably less than 90 μm. A layer thickness of more than 100 μm leads to a lower catalytic activity, in relation to the amount of catalyst used, since owing to diffusion the inner region of the catalytically active layer can no longer be reached sufficiently. The flexibility of the shaped bodies furthermore decreases, so that in some cases they cannot be processed to form corrugated or curved shaped bodies without damaging the catalytically active layer.

The above specifications of the layer thickness relate both to the layer thickness of the sprayed, as yet unleached catalytically active layer, and to the layer thickness of the leached Raney metal layer. In other words, the layer thickness does not experience any substantial change when leaching.

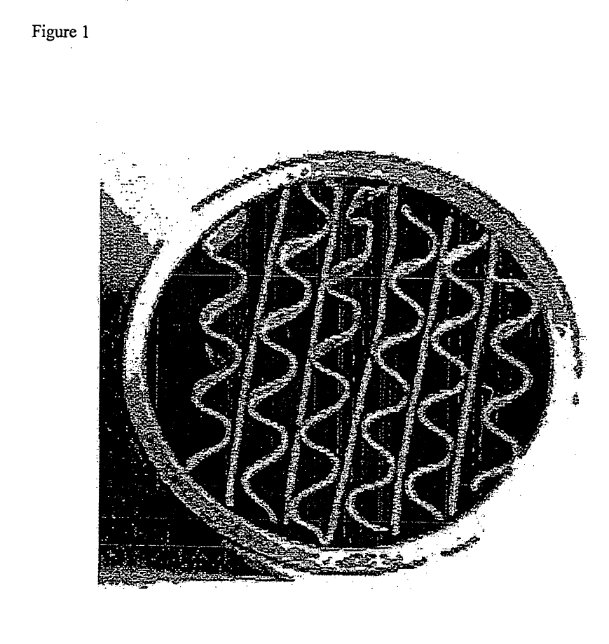

In the scope of the invention, in the context of the shaped catalyst body according to the invention, the term shaped body is intended to mean any regularly shaped three-dimensional body, particularly in contrast to an irregularly shaped powder. The shaped body is preferably a layer-like shaped body which may be straight and/or curved, for example corrugated. The shape of the shaped body according to the invention will naturally be determined by the shape of the support. According to the invention, for example, layer-shaped supports may be coated on one or both sides by thermal spraying. The layer-like shaped bodies obtained in this way can be used per se or bent, and placed in a plurality of layers above one another in order to increase the catalytically active surface area in relation to the flow cross section of a reactor. Such an embodiment is shown by way of example in FIG. 1. The shaped catalyst bodies according to the invention preferably consist of one or more layer-shaped and/or curved layer-shaped elements. A plurality of shaped catalyst bodies according to the invention may be joined together to form catalyst packing.

The shaped bodies are particularly preferably formed into the desired final shape before the catalytically inactive material is leached.

In a preferred embodiment, the supports have a compact shape essentially comprising no openings. In particular, a porous structure of the support is less preferred. In particular, the support is preferably formed by thin layers, such as foils or sheets.

The support is preferably selected from a metal or metal alloy. Particularly preferred support materials are steel, rustproof steel, stainless steel, or other similar materials.

A particularly preferred shaped body according to the invention has a support made of stainless steel, particularly preferably stainless steel with the material No 1.4767 (designation according to “Stahleisenliste”, 8th edition, pages 87, 89 and 101, ed. Verein Deutscher Eisenhüttenleute, Verlag Stahleisen mbH, Dusseldorf 1990), and the catalytically active layer is obtained by spraying a nickel/aluminium alloy (preferably with a Ni/Al atomic ratio of 10:90 to 50:50, preferably 40:60 to 20:80) preferably by plasma spraying or high-speed flame spraying.

The thickness of the support is preferably 30 to 350 μm. Smaller thicknesses lead to a lower mechanical strength of the catalyst packing. Excessive thicknesses are disadvantageous owing to the high weight.

The catalytically inactive metal, which is used to produce the shaped catalyst bodies according to the invention, is preferably selected from the group which consists of aluminium, silicon, magnesium and/or zinc. Aluminium is particularly preferred.

After co-application with-the catalytically active metal, as mentioned, the catalytically inactive metal is at least partially removed from the resulting applied layer by treatment with at least one aqueous alkali.

The alkali is preferably selected from aqueous solutions of alkali or alkaline-earth metal hydroxides, such as sodium hydroxide or potassium hydroxide.

The leaching is preferably carried out under the following conditions: particularly preferably, the leaching is carried out according to the invention with a 15 to 30 wt. % strength sodium hydroxide solution at a temperature of 60 to 110° C., at standard pressure or optionally above standard pressure. The leaching may be carried out with relative motion of the alkali with respect to the shaped body, for example by moving the shaped body in the alkali or circulating the alkali by pumping. After the alkali is added to the shaped body, an after-reaction may also take place over a period of for example up to 120 minutes.

In a manner which is known per se, a layer of the catalytically active metal with a large surface area is formed by this method. Owing to the application method according to the invention, this layer has a very high stability with respect to mechanical stress.

With the shaped catalyst body according to the invention, under the thermal spraying conditions, there is in general essentially no reaction of the sprayed metals with the support material to form intermetallic compounds.

If need be, such a reaction could be promoted by heating the support material.

In the shaped catalyst bodies according to the invention, the said metals are generally sprayed directly onto the support without an interlayer being sprayed. Such an interlayer, whether made of metal or ceramic, is not preferred according to the invention since it complicates the application method and bonding problems could rise.

The present invention also provides a method for producing the shaped catalyst body, which comprises thermally spraying at least one catalytically active metal and at least one catalytically inactive metal onto a support and subsequently removing the inactive metal(s).

The present invention also provides a reaction reactor which contains at least one shaped catalyst body according to the invention. The reactor may be any reactor suitable for carrying out heterogeneously catalysed reactions, for example a fixed bed reactor or a loop reactor, with internal or external recycling.

The invention furthermore provides a shaped body, obtainable by thermally spraying an alloy of nickel and aluminium, which contains less than 56 wt. % nickel, onto a support, the thickness of the sprayed layer being less than 0.1 mm (100 μm). The spraying is preferably carried out so that essentially no reaction takes place to form intermetallic bonds between the sprayed metals and the support. Such a shaped body constitutes an intermediate product in the production of the shaped catalyst body according to the invention, from which the shaped catalyst body according to the invention is formed by removing at least a part of the aluminium from the sprayed layer.

The invention furthermore provides the use of the shaped catalyst body according to the invention as a hydrogenation catalyst. Hydrogenation catalyst is intended to mean in particular those catalysts which are suitable for heterogeneously catalysing the addition of hydrogen to suitable compounds (hydrogenatable compounds). Such compounds are, for example, selected from carbohydrates, unsaturated fats, unsaturated hydrocarbons such as aromatics, aromatic amines, olefins, diolefins, alkynes, enamines, nitrile compounds etc., carbon monoxide, dioxide, etc.

The shaped catalyst body according to the invention is suitable in particular for hydrogenating carbohydrates, for example glucose, mannose, xylose, galactose, maltose, lactose, etc. The catalyst is particularly preferably suitable for hydrogenating glucose to form sorbitol, since the hydrogenation can be readily achieved selectively with high yields compared with known methods.

Accordingly, the invention furthermore provides the use of a shaped body, obtainable by a method which comprises thermally spraying at least one catalytically active metal and at least one catalytically inactive metal onto a support and subsequently removing the inactive metal(s), as a catalyst, in particular as a hydrogenation catalyst, preferably for hydrogenating carbohydrates such as glucose.

The invention furthermore provides a method for hydrogenating hydrogenatable compounds which uses a shaped body, obtainable by a method which comprises thermally spraying at least one catalytically active metal and at least one catalytically inactive metal onto a support and subsequently removing the inactive metal(s). The method is preferably used for hydrogenating carbohydrates.

The hydrogenation conditions depend in particular on the compound to be hydrogenated. The hydrogenation of carbohydrates is preferably carried out in water at temperatures of 40 to 200° C. and hydrogen pressures in the range of 20 to 100 bar.

The invention will be explained in more detail by the following examples.

### EXAMPLES

A) Production of the Shaped Catalyst Body:

Flat and corrugated stainless steel supports with a thickness of 0.1 mm were coated on both sides with a Ni/Al alloy layer, on average 60 μm thick, both by atmospheric plasma spraying and by high-speed flame spraying. To this end, the support surfaces were roughened before coating by jets of fine corundum with a particle size of 355/250 μm at a jet pressure of 1.5 bar. The plates treated in this way were then fixed on the sample holder and coated. In contrast to the flat supports, for which the spray gun was aligned perpendicularly to the surface, the coating angle for the corrugated plates was 70° in order to produce as uniform as possible a layer thickness over the entire surface.

The plasma spraying was carried out with a type F4 gun and the following parameters:

| | | |
|---|---|---|
| argon flow rate | 55 | l/min |
| hydrogen flow rate | 12 | l/min |
| current | 500 | A |
| voltage | 80 | V |
| power | 40 | kW |
| pistol-surface distance | 140 | mm |
| quantity of powder | 40 | g/min |

A gun of the Top Gun type was used for the high-speed flame spraying. The parameters were adjusted as follows:

| | | |
|---|---|---|
| hydrogen | 22.6 | m3/h |
| oxygen | 7.9 | m3/h |
| pistol-surface distance | 220 | mm |
| quantity of powder | 30 | g/min. |

The catalysts were activated with 25 wt. % strength sodium hydroxide solution at 50° C. and after-reaction for two hours. They were then washed with process water.

B) Hydrogenation of Glucose:

The hydrogenations were carried out under the following conditions:

10% glucose in water was hydrogenated at 40 bar of H2 and 120° C. with the activated shaped bodies. Commercially available catalysts from HC Starck were used for comparison. A slurry test (powder catalyst: Amperkat SK-NiMo 5546) and a test with tablets (Amperkat SK-Ni 5586T) were carried out in a stirred vessel (stirrer speed: 1000 rpm). The tests with the shaped bodies according to the invention (stacked corrugated and straight plates coated on both sides, L=150 mm, D=22 mm) were carried out in a loop reactor in which the following flow rates were adjusted: 550 1/h of gas and 55 l/h of liquid.

The various catalysts were compared using the rate constant (according to a pseudo-first order rate law).

Table 1 shows the test results.

TABLE 1

| Catalyst | Rate constant 1/s*kg CAT |
|---|---|
| Powder catalyst: | 0.03238 |
| Amperkat SK-NiMo 5546 | |
| Tablet catalyst: | 0.00306 |
| AMPERKAT SK-Ni 5586T | |
| Raney structure according to the invention | 0.03511 |

The tests show that the shaped bodies according to the invention actually have a slightly improved activity, in relation to the amount of catalyst. At the same time, they have significant handling advantages over powder catalysts. The shaped bodies according to the invention can be readily adapted in respect of their shape to particular (hydrogenation) processes or prefabricated for particular processes. They may also be delivered in an already activated form, e.g. in the form of replaceable elements, to customers who can transport then, store them and straightforwardly replace used catalyst elements with them. The shaped bodies according to the invention can have their size and shape adapted in any desired way to an intended (hydrogenation) process.

1. Shaped catalyst body, obtainable by a method which comprises thermally spraying at least one catalytically active metal and at least one catalytically inactive metal onto a support and subsequently removing the inactive metal(s).

2. Shaped catalyst body according to claim 1, wherein the thermal spraying is selected from the group of methods which consists of: flame spraying, for instance high-speed flame spraying; detonation spraying; plasma spraying, for instance atmospheric plasma spraying or low-pressure plasma spraying; laser spraying; arc discharge spraying and cold-gas spraying.

3. Shaped catalyst body according to claim 1 or 2, wherein an alloy which contains at least one catalytically active metal and at least one catalytically inactive metal, and optionally a promoter metal other than these metals, is sprayed.

4. Shaped catalyst body according to one of claims 1 to 3, wherein the layer thickness of the sprayed catalytically active layer is less than 100 μm.

5. Method for producing the shaped catalyst body according to one of claims 1 to 4, which comprises thermally spraying at least one catalytically active metal and at least one catalytically inactive metal onto a support and subsequently removing the inactive metal(s).

6. Reaction reactor, containing the shaped catalyst body according to one of claims 1 to 4.

7. Shaped body, obtainable by thermally spraying an alloy of nickel and aluminium, which contains less than 56 wt. % nickel, onto a support, the thickness of the sprayed layer being less than 0.1 mm.

8. Use of the shaped catalyst body according to one of claims 1 to 4 as a hydrogenation catalyst.

9. Use of a shaped body, obtainable by a method which comprises thermally spraying at least one catalytically active metal and at least one catalytically inactive metal onto a support and subsequently removing the inactive metal(s), as a catalyst.

10. Method for hydrogenating hydrogenatable compounds which uses a shaped body, obtainable by a method which comprises thermally spraying at least one catalytically active metal and at least one catalytically inactive metal onto a support and subsequently removing the inactive metal(s).
